(19) United States
(12) Patent Application Publication
(10) Pub. No.: US 2024/0053362 A1
(43) Pub. Date: Feb. 15, 2024

(54) DYNAMIC MONITORING OF E3G, LH, PdG, FSH LEVELS IN FEMALE SUBJECTS TO PREDICT HEALTH CONDITIONS

(71) Applicant: Varun AKUR VENKATESAN, Bangalore (IN)

(72) Inventors: VARUN AKUR VENKATESAN, BANGALORE (IN); SIDDARTH PATTNAIK, BANGALORE (IN); DIPANKAR DAS, BANGALORE (IN)

(21) Appl. No.: 18/266,250

(22) PCT Filed: Dec. 8, 2021
(86) PCT No.: PCT/IN2021/051149

§ 371 Date: Jun. 8, 2023

(30) Foreign Application Priority Data
Dec. 8, 2021 (IN) 202011024012

(51) Int. Cl. G01N 33/68; G01N 33/74; G01N 33/543

(52) U.S. Cl. CPC G01N 33/689; G01N 33/74; G01N 33/54388

(57) ABSTRACT

The present invention relates to a computer-implemented system [100] and method [300] for determining and monitoring multiple health conditions in a female body based on analytes present in a biological sample of said body. In an embodiment, the system [100] comprises test strips [110A-110N] coupled with a user device [130]. When said biological sample is poured on the test strip [110], the sample is pre-treated followed by reactions comprising (if) reaction between pre-treated sample and a detector reagent; (ii) reaction between each analyte of the reacted sample with corresponding biochemical reagent thereby producing a characteristic colour for each analyte. The information relating to the reactions is transmitted to the user device [130] that further determines a concentration value of each analyte based on said reactions. The user device [130] then analyses a comparison between the measured concentration value and pre-defined concentration value of each analyte to determine the fertility level.

### FIELD OF INVENTION

The present invention generally relates to predicting/determining E3G, LH, PdG and FSH LEVELS in a female subject. More particularly, the present invention relates to a hand-held system and method for dynamically determining levels/concentrations of a number of analytes E3G, LH, PdG and FSH present in a biological sample obtained from the female subject to obtain fertility level of the female subject and

### BACKGROUND OF THE INVENTION

In current scenario, continuing efforts are made to understand a complex process of fertility in a female body. Fertility management is crucial for a female body due to numerous reasons inter alia achieving/avoiding pregnancy, vitro fertilization, detecting/managing/treating menopause, and other health conditions/disorders. In the process of monitoring/managing fertility level in a female body, the very first step is to attain information on fertility status, fertile phase in the cycle (ovulation), etc.

Therefore, considering the criticality of fertility management, various methods/devices have been proposed in the art in order to predict/treat fertility level and to provide effective and reliable birth control solutions. Few of the existing/conventional techniques solely rely on variation in few parameters during ovulation cycle. One of the major drawbacks inherent in such techniques is inconsistency in parameter variation from one female to another, and certainly from one cycle to another cycle in the same female body which in turn makes the observations/data unreliable and inaccurate for practical use.

Other conventional fertility testing methods involve measuring concentration/level of parameters/analytes such as Estradiol glucuronide (E3G) and Luteinizing hormone (LH) for assessment of fertility status, since it is known that high levels of estradiol precede LH surge in the female body and LH surge triggers ovulation (follicle rupture). Therefore, high E3G levels and peak LH level indicate high fertility and peak fertility respectively. Further, Pregnanediol glucuronide (PdG) is a urine metabolite of progesterone (secreted by the Corpus Luteum) and therefore indicates occurrence of ovulation. Furthermore, Progesterone supports the endometrium, thus allowing pregnancy to continue. In a particular ovulation cycle, if the female body does not conceive, progesterone levels fall. Alternatively, if the female conceives, the progesterone levels are maintained throughout the pregnancy period. In addition, a quick decline in PdG levels during pregnancy is an indicator of putative miscarriage. Once the PdG falls (shown in FIG. 4A), follicle stimulating hormone (FSH) increases enabling maturation of the follicles and a new cycle begins. However, the conventional/existing technologies fail to effectively provide results/data for multiple scenarios/tests using a single test to predict, detect or establish fertility test in the female. Moreover, the female/user is required to perform individual tests for monitoring each of the follicle maturation, follicle growth (fertility), follicle rupture (ovulation) and successful conception. Thus, the existing technologies significantly face performance issues, are not only time consuming but are also frustrating for the user.

Accordingly, in view of the aforementioned limitations inherent in the existing fertility testing methods, there exists a need to provide a single test to determine multiple parameters/analytes affecting the fertility test. More particularly, there exists a need of a computer-implemented system and method for personal, home-based and/or point-of-care determining fertility level in a female body based on a plurality of analytes present in a biological sample of female/user.

### SUMMARY OF THE INVENTION

In view of the limitations of the existing conventional technologies/systems as discussed above, it is evident that there arises a need for developing an efficient and effective system and method for substantially overcoming said limitations. It is, therefore, an objective of the present invention to provide a computer-implemented method and system (test kit and user device) to determine (personal, home-based and/or point-of-care) fertility level in a female body through a single test/process. Another objective of the present invention is to simultaneously monitor the fertility level in the female body through a user device being coupled to the test strip (on which test is conducted). Yet another objective of the present invention is to determine absolute and relative concentrations of analytes in the biological sample and determine fertility test.

More particularly, the present invention relates to a computer-implemented method for determining fertility level in a female body based on a plurality of analytes present in a biological sample from the female body. The method comprising: pre-treating, at a test strip, the biological sample received from the female body, wherein the plurality of analytes present in the biological sample are Oestrone-3-glucuronide (E3G), Leuteinizing Hormone (LH), PdG and FSH; reacting said pre-treated biological sample with a detector reagent comprising one or more detector antibodies, wherein said detector reagent is present in one or more conjugate pads, and each of the plurality of analytes of the reacted biological sample further flows to corresponding detecting zone; reacting each of the plurality of analytes with corresponding biochemical reagent at the corresponding detecting zone producing a characteristic colour on one or more control lines for each of the plurality of analytes, wherein the corresponding biochemical reagent is covalently attached to magnetic and electronically charged labels; determining, by a user device, a concentration value of each of the plurality of analytes based on the reactions of step 3 and step 2; and analysing, by the user device, a comparison between the measured concentration value and pre-defined concentration value of each of the plurality of analytes to determine the fertility level.

Further, in an embodiment of the present invention, the method comprises transmitting information to the user device, wherein said information comprises the reaction of pre-treated biological sample with the detector reagent, the reaction of each of the plurality of analytes with the corresponding biochemical reagent and the characteristic color for each of the plurality of analytes.

Further, in an embodiment of the present invention, the method comprises monitoring the fertility level using the handheld device.

Additionally, in another aspect, the present invention relates to a handheld computer-implemented system for determining fertility level in a female body based on a plurality of analytes present in a biological sample from the female body. The system comprising (i) a plurality of test strips comprising: a sample receiving area for receiving for receiving and pre-treating the biological sample from the female body, wherein the plurality of analytes present in the biological sample are E3G, LH, PdG and FSH, one or more conjugate pads provided with a detector reagent comprising one or more detector antibodies, wherein the detector reagent is reacted with the pre-treated biological sample, a plurality of detecting zones each provided with a corresponding biochemical regent for each of the plurality of analytes, wherein each of the plurality of analytes is reacted with corresponding biochemical reagent at the corresponding detecting zone producing a characteristic colour on one or more control lines, and the corresponding biochemical reagent is covalently attached to magnetic and electronically charged labels, and the one or more control lines for representing the characteristic color for each of the plurality of analytes; and (ii) a user device coupled with the plurality of test strips, the user device comprising: a sensing unit configured to scan the plurality of test strips for receiving information from the plurality of test strips, a quantification unit for each of the plurality of analytes to determine a concentration value of each of the plurality of analytes based on said reactions, an analytic unit configured to analyse said concentration unit and compare the measured concentration value with pre-defined concentration value of each of the plurality of analytes, and a diagnostic unit configured to determine the fertility level based on said comparison.

The afore-mentioned objectives and other aspects of the embodiments herein will be better appreciated and understood when considered in conjunction with the following description and the accompanying drawings. It is understood that the following descriptions, while indicating preferred embodiments and numerous specific details thereof, are given by way of illustration and not of limitation. Many changes and modifications may be made within the scope of the embodiments herein without deviating from the scope of the invention. Therefore, this section is intended only to introduce certain objectives and aspects of the present invention, and is therefore, not intended to define/outline key features or scope of the subject matter of the present invention.

### BRIEF DESCRIPTION OF DRAWINGS

The drawings mentioned in this section disclose exemplary embodiments of the claimed system and method. Detailed description of well-known components and processing techniques are omitted to not unnecessarily obscure the embodiments herein. Further, the components/modules/units and steps of a process are assigned reference numerals that are used throughout the description to indicate the respective components and steps. Other objects, features, and advantages of the present invention will be apparent from the following description when read with reference to the accompanying drawings.

FIG. 1 illustrate system architecture of a computer-implemented system of the present invention for determining fertility level in a female body based on a plurality of analytes present in a biological sample from the female body according to an embodiment herein;

FIG. 2 (A-I) illustrate different embodiments/possibilities for test strips in accordance with the present invention, according to an embodiment herein;

FIG. 3 illustrates a method for determining fertility level in a female body based on a plurality of analytes present in a biological sample from the female body, according to an embodiment herein; and

FIG. 4A-4B illustrates a number of plots obtained for different conditions for different hormones over a number of days, according to an embodiment herein.

### DETAILED DESCRIPTION

This section is intended to provide explanation and description of various possible embodiments of the present invention. The embodiments used herein, and the various features and advantageous details thereof are explained more fully with reference to non-limiting embodiments illustrated in the accompanying drawings and detailed in the following description. The examples used herein are intended only to facilitate an understanding of ways in which the embodiments herein may be practiced and to further enable the person skilled in the art to practice the embodiments used herein. Also, the examples/embodiments described herein should not be construed as limiting the scope of the embodiments herein. Corresponding reference numerals indicate corresponding parts throughout the drawings.

The present invention relates to a system and method for determining fertility level in a female body based on a plurality of analytes present in a biological sample from the female body. When a user wishes to undertake a fertility test, the user uses a test strip to determine fertility level. Further, when a biological sample of the user flows onto the test strip, various zones/areas of the test strip comprises antibodies, regents and other chemical substances are configured to react with the biological sample and more specifically with the analytes present in the biological sample. Information relating to said reaction is further transmitted to the user device coupled with the test strip. The user device is then configured to analyse the test results and said information and accordingly determine the fertility level and check whether the female body is in the phase of fertility/ovulation. The present invention also encompasses monitoring the fertility level based on the analytes determined.

As used herein, the biological sample refers any bodily fluid or fluid type substance from a user to determine the presence, absence, levels/concentration of any hormones, analytes, metabolites, minerals, etc. in the subject's body. The biological sample is one of sweat, urine, blood, blood serum, semen, breast milk, saliva, blood plasma, tears, mucus, cerebrospinal fluids, saliva, amniotic fluid, vaginal lubrication fluids, pus, lymph, bile, synovial fluid, aqueous humour, phlegm, gastric acid, pre-ejaculate, colostrum and other such fluids as may be obvious to a person skilled in the art. Further, the biological sample comprises a plurality of analytes including, but not limiting to, Estradiol glucuronide (E3G), Luteinizing Hormone (LH), Pregnanediol Glucuronide (PdG) and Follicle-stimulating hormone (FSH). The terms ‘biological sample’ and ‘sample’ may refer to similar meaning/interpretation and may be interchangeably used throughout the specification. The terms “level” in context of analytes refer to concentration or amount in blood or the biological sample chosen. The term “level” in context of fertility and other such health conditions refers to their presence or absence (aka high or low) of a health condition.

As used herein, the test strip refers to a medium or base or any paper-based device on which a biochemical reagent and other antibodies are integrated/affixed/immobilized to react with an analyte present in the biological sample taken from the user. In an embodiment, the test strip may be immunoassay strips, while in another embodiment, the test strip may be immunochromatographic strips. The terms ‘test strip’ and ‘strip’ may refer to similar meaning/interpretation and may be interchangeably used throughout the specification. Further, the test strip comprises a plurality of assays for measuring the relative or absolute concentrations of the analytes in the biological sample.

As used herein, the user device refers to any computing device capable of reading/scanning/detecting the test strip and detecting desired analyte's presence or absence or levels in the biological sample. The user device may be a smartphone, a computer, a laptop, a stand-alone system, a dedicated metre or device and any such device as may be obvious to a person skilled in the art. Further, the user device is coupled/connected with the test strip for the objective of scanning/reading the test strip. Furthermore, the user of the user device may be a female, a patient, doctor and any such person as may be obvious to a person skilled in the art. The terms ‘user’, ‘patient’, ‘female’, ‘body’ and ‘subject’ may refer to similar meaning/interpretation and may be interchangeably used throughout the specification.

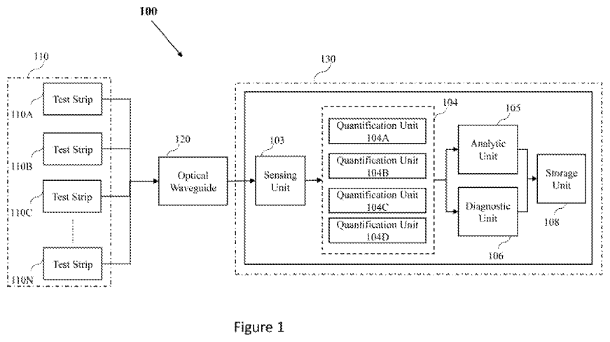

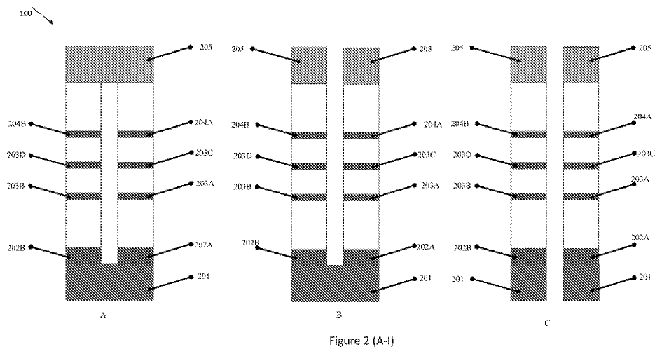

Further, it is to be understood that the terms such as connect, couple and other similar terms include a physical connection (such as a wired/wireless connection), a logical connection or a combination of such connections as may be obvious to a person skilled in the art. FIG. 1 illustrates a system overview of a system for determining levels of PdG, FSH, E3G and LH in a biological sample obtained from a female subject and subsequently monitoring the several health conditions level in accordance with an embodiment of the present invention. In an embodiment, the health conditions include fertility level, follicle health, ovarian reserve, egg quality, follicular growth, corpus luteum integrity, miscarriage, viability of embryo, early diagnosis of pregnancy, miscarriage, menopause, PMS severity, period of intercourse, right time to stimulate during in-vitro fertilisation procedure.

More particularly, a method for determining a plurality of health conditions for a female subject is provided. The method includes determining concentrations or levels of analytes selected from Estradiol glucuronide (E3G), Luteinizing Hormone (LH), Pregnanediol Glucuronide (PdG) and/or Follicle-stimulating hormone (FSH) and human Chorionic Gonadotropin (hCG); mapping concentration of at least one analyte with respect to another in a luteal or follicular phase; and predicting a health condition of the female subject. In an embodiment, rate of change of concentrations/levels of the hormones is determined or monitored.

More particularly, the system [100] comprises a plurality of test strips [110A-110N], and a user device [130], wherein the information is directly and accurately transmitted from the plurality of test strips [110A-110N] to the user device through an optical waveguide [120]. Further, the test strips [110A-110N] comprises a sample receiving pad/area [201], one or more conjugate pads [202A, 202B], a plurality of detecting zones [203A, 203B, 203C, 203D] and one or more control lines [204A, 204B, 204C]. Furthermore, the user device comprises a sensing unit [103], quantification units [104A, 104B, 104C, 104D], an analytics unit [105] and a diagnostic unit [106], wherein each unit is coupled/connected to each other in accordance with the present invention.

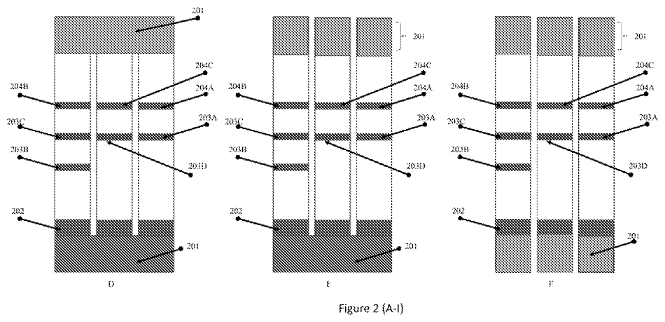

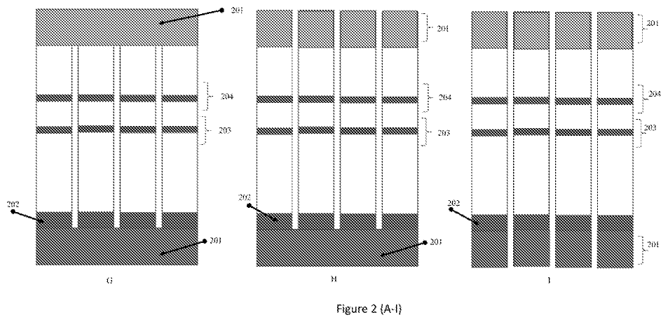

Further, as illustrated in FIG. 1 and FIG. 2 (A-I), if the user wishes to determine fertility level, the user is required to put/flow/pass on the biological sample onto the sample receiving area [201] of the test strip [110], wherein the sample receiving area [201] is a porous membrane comprising a binding reagent having capability to bind to each of the plurality of analytes.

Subsequently, the pre-treated sample flows to the one or more conjugate pads [202A, 202B] where the detector reagent comprising one or more detector antibodies is reacted with said pre-treated sample (i.e. with the plurality of analytes present in the sample). The one or more conjugate pads [202A, 202B] comprises at least one of glass fibre-based polymers, woven cellulose fibre polymers and non-woven cellulose fibre polymers. In an embodiment, the one or more conjugate pads [202A, 202B] are integrated with the sample receiving area [201], while in another embodiment, the one or more conjugate pads [202A, 202B] are not integrated with the sample receiving area and provided separately on the test strip [110].

The test strip is provided with a plurality of detecting zones [203A, 23B, 203C, 203D], wherein each detecting zone is configured for one analyte, and the detecting zones [203A-203D] comprise corresponding biochemical reagent/s. Pursuant to the reaction at the one of more conjugate pad [202A, 202B], each of the plurality of analytes of the reacted biological sample (combination of the biological sample and the one or detector antibodies) further reacts with the corresponding biochemical reagent at the corresponding detecting zone [203A-203D]. In an exemplary embodiment, 203A is detecting zone for FSH, 203B refers to detecting zone for PdG, 203C is detecting zone for LH, and 203D is detecting zone for E3G i.e. reaction of FSH with the corresponding biochemical reagent occurs at detecting zone 203A and so on. This reaction of the analytes with the biochemical reagent produces a characteristic colour on one or more control lines [204A, 204B, 204C] for each of the plurality of analytes, wherein the corresponding biochemical reagent is covalently attached to magnetic and electronically charged labels. In an embodiment, said labels produce signals through either chemical or physical means, such as being optically detectable. Said labels include enzymes and substrates, chromogens, catalysts, fluorescent compounds, chemiluminescent compounds, electroactive species, dye molecules, radioactive labels and particle labels. In another embodiment, the particle labels may include magnetic or electronically charged labels, which can be detected by magnetic or electrochemical means. In a preferred embodiment, the optically detectable labels include colloidal metallic particle labels and dye-laden particles.

Further, when the user of the user device [130] scans the image of the reaction and/or the characteristic colours, the information relating to the reaction of pre-treated biological sample with the detector reagent, the reaction of each of the plurality of analytes with the corresponding biochemical reagent and the characteristic color for each of the plurality of analytes is transmitted to the sensing unit [103] of the user device [130] through the optical waveguide [120]. In an embodiment, the optical waveguide [120] is configured to collect light optimally and project it on the test strip. In an embodiment, the optical waveguide [120] is present in the user device [130] to steer beam from said test strips [110] in desired direction. In another embodiment, the optical waveguide includes a transparent optical block, a thin layer of highly reflective prism [that steers the beams], mirror to direct the incoming light to the camera, and a plano-convex lens that couples the camera/sensing unit [103] of the user device with the optical waveguide [120].

The sensing unit [103] is therefore configured to scan the image/reaction conducted on the test strip [110] and receive said information from the test strip [110]. Said information is then internally communicated to the quantification units [104A, 104B, 104C, 104D] for determining concentration values for each of the plurality of analytes. In particular, the user device [130] comprises a quantification unit [104A-104D] for individually determining concentration value for each of the analytes. For instance, quantification unit 104A is configured to determine the concentration value of FSH and so on. Thus, the quantification units [104A-104D] are configured to determine/quantify concentration value of each of the plurality of analytes, wherein the concentration value is relative concentration and absolute concentration.

Based on the concentration values determined, the analytic unit [105] is configured to analyse said concentration values and compare the measured concentration value of each analyte with pre-defined concentration value, wherein the pre-defined concentration values are defined by the user or the system [100]. In an embodiment, the pre-defined values for the plurality of analytes are different while in another embodiment, the pre-defined values for the plurality of analytes are similar. In an alternative embodiment, the analytic unit [105] is configured to compare the characteristic color for each of the plurality of analytes on the test strip [110] with the pre-defined concentration value of each of the plurality of analytes, wherein the pre-defined concentration value for each analyte is defined by the user or the system [100]. In an embodiment, the analytic unit [105] is present in the user device [130] while in another embodiment, the analytic unit [105] is present on a remote server connected to the user device [130].

Based on said analysis (comparison of one of the concentration values and the characteristic colors), the diagnostic unit [106] is configured to determine a number of health conditions as mentioned in the foregoing Further, the present invention also encompasses monitoring the fertility level through the diagnostic unit [106], wherein said monitoring may be periodic or non-periodic.

Furthermore, the present invention relates to various embodiments of the test strip [110] comprising varied number of lateral flow assays. More particularly and as illustrated in FIG. 2 (A-I), the assay design comprises n number of lateral flow assays. In first embodiment, the assay design consists of four lateral flow assays, wherein one assay is for one corresponding analyte (i.e. E3G, LH, PdG and FSH). In second embodiment, the assay design comprises two lateral flow assays, wherein one flow assay is for measurement of two analytes in a competitive assay format (E3G and PdG) and the other assay is for measurement of two analytes in a sandwich assay format (LH and FSH). In third embodiment, the assay design comprises three lateral flow assays, wherein one assay is for measuring both the analytes in a competitive or sandwich manner and the other two assays are for measuring remaining analytes. Further, referring to all the embodiments illustrated in FIG. 2, the flow path of the different lateral flow assays is either same or different. Furthermore, the one or more control lines [204A, 204B, 204C] correspond for at least one of multiplexed competitive assay, individual competitive assay, multiplexed sandwich assay and individual sandwich assay.

Furthermore, in an embodiment, the user device [130] also comprises a storage unit [108] configured to store said information, user's details and any such detail as may be obvious to a person skilled in the art. In an alternative embodiment, the storage unit [108] is at server. As used herein, the term storage unit and similar terms such as datastore, database, cache, memory refer to similar interpretation and may be used interchangeably throughout the specification. Further, the storage unit [108] may refer to a volatile memory, a non-volatile memory, or a combination thereof as may be obvious to a person skilled in the art.

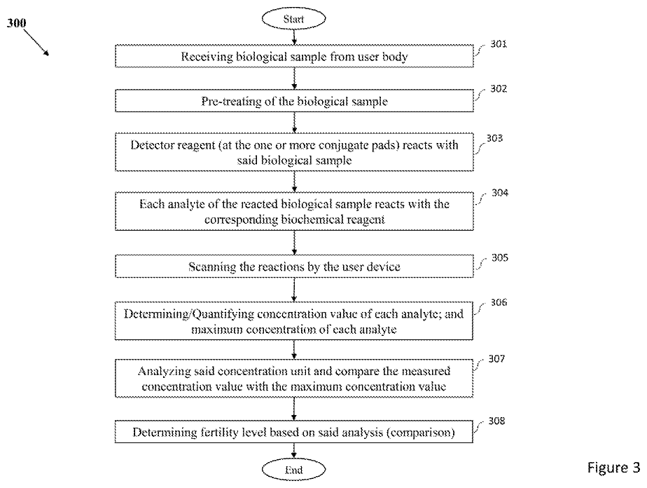

Similarly, FIG. 3 illustrates a method overview [300] of the present invention for determining levels of E3G, PdG, LH and FSH and predicting or diagnosing a health condition. The method [300] comprises a series of following steps to accomplish the objective encompassed by the present invention:

At step 301, the sample receiving area [201] receives the biological sample from the user body if the user wishes to determine fertility level.

At step 302, the plurality of analytes (E3G, LH, PdG and FSH) present in said biological sample are pre-treated. Subsequently, the pre-treated biological sample flows to the one or more conjugate pads [202A, 202B].

At step 303, the detector reagent (at the one or more conjugate pads [202A, 202B]) comprising one or more detector antibodies is reacted with said biological sample (i.e. with the plurality of analytes present in the sample).

At step 304, each of the plurality of analytes of the reacted biological sample reacts with the corresponding biochemical reagent at the corresponding detecting zone [203A-203D]. In the preferred embodiment, the reacted biological sample is a combination of the biological sample and the one or detector antibodies. This reaction of the analytes with the biochemical reagent produces a characteristic colour on one or more control lines [204A, 204B, 204C] for each of the plurality of analytes, wherein the corresponding biochemical reagent is covalently attached to magnetic and electronically charged labels.

At step 305, the user of the user device [130] scans the image of the reaction and/or the characteristic colours the information relating to the reaction of pre-treated biological sample with the detector reagent. Pursuant to the scanning, the reaction of each of the plurality of analytes with the corresponding biochemical reagent and the characteristic color for each of the plurality of analytes are transmitted to the sensing unit [103] of the user device [130] through the optical waveguide [120]. The sensing unit [103] is therefore configured to scan the image/reaction conducted on the test strip [110] and receive said information from the test strip [110]. Said information is then internally communicated to the quantification units [104A, 104B, 104C, 104D] for determining concentration values for each of the plurality of analytes.

At step 306, the quantification units [104A-104D] are configured to determine/quantify concentration value of each of the plurality of analytes, wherein the concentration value is relative concentration and absolute concentration.

At step 307, the analytic unit [105] is configured to analyse said concentration unit and compare the measured concentration value with pre-defined concentration value of each of the plurality of analytes.

At step 308, the diagnostic unit [106] is configured to determine the health conditions based on said analysis (comparison of one of the concentration values and the characteristic colors) and a request placed by a user. Further, the present invention also encompasses monitoring the fertility level through the diagnostic unit [106], wherein said monitoring may be periodic or non-periodic.

In an embodiment, several health conditions are determined by comparing concentrations or levels of two or more of the analytes selected from E3G, PdG, LH and FSH.

In an aspect, a method of determining follicle health or ovarian reserve or egg quality in a female subject is provided by using methods and systems described above. The above systems and methods may be deployed to study relation between several hormones or all hormones and determine a health condition. In an embodiment, the method includes monitoring FSH and E3G on third day of the menstruation cycle, such that a high FSH and a low E3G reading determines egg quality or follicle health in the female subject. Further, a healthy follicular growth and normal behaviour in the female subject is determined by monitoring E3G pattern, which usually fluctuates every 36-38 hours with a constant E3G rise till LH attains peak. Further, the egg quality as well as corpus luteum integrity is determined by analysing level of LH surge on the peak day of the menstrual cycle while monitoring the time taken by progesterone concentration to rise. The corpus luteum is an endocrine structure that is involved in ovulation and early pregnancy. During ovulation, secondary follicles are formed from primary follicles followed by formation of mature vesicular follicle. At ovulation, the follicle ruptures expelling the ovum into the fallopian tube. The corpus luteum produces oestrogen and progesterone to maintain conditions for implantation and if not implanted, the corpus luteum involutes and turns into a corpus albicans. In a further embodiment, the viability of embryo or risk of miscarriage after conception may be determined by analysing peak PdG levels in luteal phase and rate of rise of PdG rise after LH peak.

In a further embodiment, an early diagnosis of pregnancy is made using systems and methods as discussed in the foregoing. In an embodiment, if PdG concentration is more than 15 ug/ml along with the hCG concentration being higher than 10 mIU/ml, a pregnancy is confirmed. In an embodiment, a miscarriage may be diagnosed if the hCG concentration remains high while PdG's concentration drops. A very high FSH concentration followed by low E3G in aged female subjects serves as marker for predicting menopause or depleting ovarian reserve. Further, in another embodiment, high estrogen and low/fluctuating progesterone are used as markers of potential periods of severe PMS.

Further, the system and methods mentioned in the foregoing may also be used to determine contraception. In an embodiment, a method of identifying days of low fertility is provided. The method includes monitoring FSH and E3G in follicular phase and PdG concentration in luteal phase. The days of low fertility show high FSH and low E3G in the follicular phase, which is a low fertility window; whereas in luteal phase if PdG's concentration falls by more than Eng/ml, this is predicted as low fertility window.

Similarly, a method of monitoring in-vitro fertilisation (IVF) is provided. The method includes determining a time or period for stimulation. After recruitment to a study for IVF, the patients are monitored for estrogen rise before retrieval of egg. In IVF cycles aiming at egg retrieval, ovulation induction is done by providing hMG shots (LH+FSH) that leading to growth of follicles. The estrogen levels, however, need to be monitored so that they do not cross a certain threshold early in the cycle (around 2000 ng/ml). Once the E3G levels reach around 2000 ng/ml, hCG shot is given in order to induce ovulation and retrieve eggs. A dual track E3G test strip can be used to monitor
a. The lower levels of E3G where induction needs to be done or for normal cycles (with the first strip with a lower range of detection);
b. Prevent higher levels of E3G while inducing (with the second strip with a higher range of detection).

Further, while implanting the in-vitro fertilized egg, PdG concentration needs to be monitored in order to make sure the endometrium is ready for implantation.

Thus, the present invention relates to computer-implemented system and method for determining fertility level based on concentration of the plurality of analytes in the biological sample. Also, the present invention encompasses monitoring the fertility level in the user (body). In addition, the present invention is applicable to various fields/uses such as health, pregnancy, menopause, and such health conditions/disorders where the fertility test is required and is obvious to a person skilled in the art.

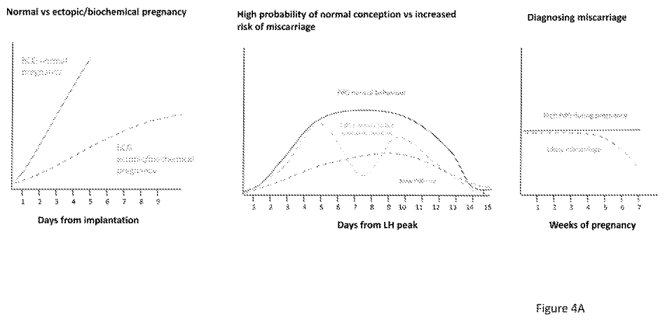

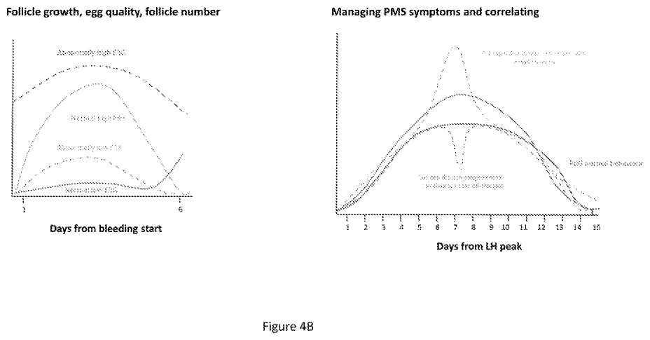

FIG. 4A-4B illustrates a number of plots obtained for different conditions for different hormones over a number of days, according to an embodiment herein. One of the plot shows the hormonal levels i.e. of hCG as determined during ectopic and normal pregnancy, thus allowing prediction of whether the pregnancy is normal or ectopic.

In an further embodiment, a method of predicting follicle growth, egg quality and follicle number is provided. The method includes determining E3G and FSH levels following the menstruation over the next few days, preferably 6 [as shown in FIG. 4B]. In another embodiment, a method for managing PMS symptoms is provided. The method includes determining E3G levels, whose dominance shows up in mood changes, whereas sudden drop in progesterone also results in mood shifts.

The term exemplary is used herein to mean serving as an example. Any embodiment or implementation described as exemplary is not necessarily to be construed as preferred or advantageous over other embodiments or implementations. Further, the use of terms such as including, comprising, having, containing and variations thereof, is meant to encompass the items/components/process listed thereafter and equivalents thereof as well as additional items/components/process.

It will be apparent that according to one aspect of this embodiment, one or more computer algorithms/processes for determining fertility level need not reside on a single computer or processor but may be distributed in a modular fashion amongst a number of different computers or processors to implement various aspects of the present invention.

Although the subject matter is described in language specific to structural features and/or acts, it is to be understood that the subject matter defined in the claims is not necessarily limited to the specific features or process as described above. In fact, the specific features and acts described above are disclosed as mere examples of implementing the claims and other equivalent features and processes which are intended to be within the scope of the claims.

Further, the subject matter of example embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to system and method for generating and monitoring the smart contract between two or more users.

We claim:

1. A method for determining a plurality of health conditions for a female subject comprising:
determining concentrations or levels, and/or rate of change in concentration or levels of analytes selected from one or more of Estradiol glucuronide (E3G), Luteinizing Hormone (LH), Pregnanediol Glucuronide (PdG), Follicle-stimulating hormone (FSH), and human chorionic gonadotropin (hCG);
mapping concentration of at least one analyte with respect to a phase of the cycle; and
predicting a health condition of the female subject.

2. The method as claimed in claim 1, wherein the plurality of health conditions are selected from fertility level, follicle health, ovarian reserve, egg quality, follicular growth, corpus luteum integrity, miscarriage, viability of embryo, early diagnosis of pregnancy, miscarriage, menopause, PMS severity, period of intercourse, right time to stimulate during in-vitro fertilisation procedure.

3. The method as claimed in claim 2, further comprising determining doubling time of hCG to determine whether pregnancy is normal, ectopic or biochemical.

4. The method as claimed in claim 2, further comprising monitoring PdG levels or concentrations for 2 to 3 days after LH surge; wherein when the concentration of PdG is more than twice a base level, early ovulation is predicted.

5. The method as claimed in claim 1, wherein the health condition pertaining to a fertility level of the female subject is predicted by measuring concentration of each of the E3G, FSH, LH, PdG and hCG and analysing a comparison between measured concentration value and pre-defined concentration value of each of the E3G, FSH, LH, PdG and hCG.

6. The method as claimed in claim 1, wherein the health conditions pertaining to follicle health, ovarian reserves and egg quality in the female subject is predicted by comparing FSH levels with E3G on third day of bleeding period of the menstrual cycle of the subject.

7. The method as claimed in claim 1, wherein the health conditions pertaining to corpus luteum integrity, viability of embryo and risk of miscarriage is predicted based on surge of LH levels on peak day of the menstrual cycle followed by rate of rise of levels of PdG, and further by determining peak PdG levels in luteal phase and rate of rise in PdG levels after LH's peak concentration; wherein PdG value increases steadily within first 3-5 days to more than 10 ug/ml.

8. The method as claimed in claim 1, wherein the health conditions pertaining to early diagnosis of pregnancy in the female subject is predicted by determining whether PdG level is greater than 15 ug/ml and whether hCG levels is greater than 10 mIU/ml.

9. The method as claimed in claim 1, wherein the health conditions pertaining to a PMS severity in the female subject is predicted by monitoring E3G and PdG levels; wherein high E3G and low PdG or fluctuating PdG are markers for severe PMS.

10. The method as claimed in claim 1, wherein the health conditions pertaining to low fertility window in the female subject is predicted by monitoring FSH and E3G levels in follicular phase and PdG levels in luteal phase, wherein high FSH and low E3G indicates low fertility window in follicular phase and wherein PdG levels fall more than 6 ng/ml in luteal phase indicating low fertility window.

11. The method as claimed in claim 1 further comprising:
pre-treating, at a test strip [110], the biological sample received from the female body, wherein the plurality of analytes present in the biological sample are E3G, LH, PdG and FSH;
reacting said pre-treated biological sample with a detector reagent comprising one or more detector antibodies, wherein said detector reagent is present in one or more conjugate pads [202A, 202B], and each of the plurality of analytes of the reacted biological sample further flows to corresponding detecting zone [203A, 203B, 203C, 203D];
reacting each of the plurality of analytes, of the reacted biological sample, with corresponding biochemical reagent at the corresponding detecting zone [203A-203D] producing a characteristic colour on one or more control lines [204A, 204B, 204C] for each of the plurality of analytes, wherein the corresponding biochemical reagent is covalently attached to magnetic and electronically charged labels;
determining, by a user device [130], a concentration value of each of the plurality of analytes based on the reactions of step 3 and step 2; and
analysing, by the user device [130], a comparison between the measured concentration value and pre-defined concentration value of each of the plurality of analytes to determine the fertility level.

12. A method [300] for determining fertility level in a female subject based on a plurality of analytes present in a biological sample from the female subject, the method [300] comprising:
pre-treating, at a test strip [110], the biological sample received from the female body, wherein the plurality of analytes present in the biological sample are E3G, LH, PdG and FSH;
reacting said pre-treated biological sample with a detector reagent comprising one or more detector antibodies, wherein said detector reagent is present in one or more conjugate pads [202A, 202B], and each of the plurality of analytes of the reacted biological sample further flows to corresponding detecting zone [203A, 203B, 203C, 203D];
reacting each of the plurality of analytes, of the reacted biological sample, with corresponding biochemical reagent at the corresponding detecting zone [203A-203D] producing a characteristic colour on one or more control lines [204A, 204B, 204C] for each of the plurality of analytes, wherein the corresponding biochemical reagent is covalently attached to magnetic and electronically charged labels;
determining, by a user device [130], a concentration value of each of the plurality of analytes based on the reactions of step 3 and step 2; and
analysing, by the user device [130], a comparison between the measured concentration value and pre-defined concentration value of each of the plurality of analytes to determine the fertility level.

13. The method [300] as claimed in claim 1, further comprising transmitting information to the user device [130], wherein said information comprises the reaction of pre-treated biological sample with the detector reagent, the reaction of each of the plurality of analytes with the corresponding biochemical reagent and the characteristic color for each of the plurality of analytes.

14. The method [300] as claimed in claim 1, further comprising monitoring the fertility level using the user device [130].

15. The method [300] as claimed in claim 1, wherein said biological sample is one of blood, urine, plasma and serum.

16. The method [300] as claimed in claim 1, wherein the reacted biological sample refers to a combination of the biological sample and the one or detector antibodies.

17. The method [300] as claimed in claim 1, wherein said concentration value is relative concentration and absolute concentration.

18. A handheld computer-implemented system [100] for determining fertility level in a female body based on a plurality of analytes present in a biological sample from the female body, the system [100] comprising
a plurality of test strips [110A-110N] comprising:
a sample receiving area [201] for receiving for receiving and pre-treating the biological sample from the female body, wherein the plurality of analytes present in the biological sample are E3G, LH, PdG and FSH,
one or more conjugate pads [202A, 202B] provided with a detector reagent comprising one or more detector antibodies, wherein the detector reagent is reacted with the pre-treated biological sample,
a plurality of detecting zones [203A, 203B, 203C, 203D] each provided with a corresponding biochemical regent for each of the plurality of analytes, wherein each of the plurality of analytes, of the reacted biological sample, is reacted with corresponding biochemical reagent at the corresponding detecting zone [203A-203D] producing a characteristic colour on one or more control lines [204A, 204B, 204C], and the corresponding biochemical reagent is covalently attached to magnetic and electronically charged labels, and
the one or more control lines [204A, 204B, 204C] for representing the characteristic color for each of the plurality of analytes; and
a user device [130] coupled with the plurality of test strips [110], the user device [130] comprising:
a sensing unit [103] configured to scan the plurality of test strips [110] for receiving information from the plurality of test strips [110],
a quantification unit [104] for each of the plurality of analytes to determine a concentration value of each of the plurality of analytes based on said reactions,
an analytic unit [105] configured to analyse said concentration unit and compare the measured concentration value with pre-defined concentration value of each of the plurality of analytes, and
a diagnostic unit [106] configured to determine the fertility level based on said comparison.

19. The system [100] as claimed in claim 8, wherein the plurality of test strips [110] are immunochromatographic assay strips.

20. The system [100] as claimed in claim 8, wherein the sample receiving area [201] is a porous membrane comprising a binding reagent having capability to bind to each of the plurality of analytes.

21. The system [100] as claimed in claim 8, wherein the one or more conjugate pads [202A, 202B] are integrated with the sample receiving area [201].

22. The system [100] as claimed in claim 8, wherein the one or more conjugate pads [202A, 202B] are not integrated with the sample receiving area [201].

23. The system [100] as claimed in claim 8, wherein the one or more conjugate pads [202A, 202B] comprises at least one of glass fibre-based polymers, woven cellulose fibre polymers and non-woven cellulose fibre polymers.

24. The system [100] as claimed in claim 8, wherein the one or more control lines [204A, 204B, 204C] correspond for at least one of multiplexed competitive assay, individual competitive assay, multiplexed sandwich assay and individual sandwich assay.

25. The system [100] as claimed in claim 8, wherein said information comprises the reaction of pre-treated biological sample with the detector reagent, the reaction of each of the plurality of analytes with the corresponding biochemical reagent and the characteristic color for each of the plurality of analytes

26. The system [100] as claimed in claim 8, wherein the diagnostic unit [106] is further configured to monitor the fertility level.
